(19) United States
(12) Patent Application Publication
(10) Pub. No.: US 2012/0172905 A1
(43) Pub. Date: Jul. 5, 2012

(54) Tissue Removal Apparatus and Method of Manufacturing Same

(75) Inventors: Natasha Lee Shee, Maple (CA); Neil Godara, Milton (CA); Padina Pezeshki, Toronto (CA); Caitlyn Paget, Toronto (CA)

(73) Assignee: Kimberly-Clark, Inc., Mississauga (CA)

(21) Appl. No.: 12/981,615

(22) Filed: Dec. 30, 2010

(51) Int. Cl. A61B 17/32; B21K 5/02

(52) U.S. Cl. 606/180; 76/115

(57) ABSTRACT

An apparatus is disclosed for removing tissue from a patient's body. The apparatus comprises an elongate member defining a lumen and a tissue removal member disposed within the lumen. The tissue removal member comprises a flexible shaft formed from a plurality of strands, and a coil that is helically disposed around the shaft. The coil is substantially in contact with the shaft along a length of the shaft. The coil forms a plurality of outwardly extending projections and each pair of the plurality of outwardly extending projections defines a spacing therebetween. Upon rotation of the tissue removal member within the elongate member, material is conveyed through the lumen of the elongate member. A related method of manufacturing a tissue removal member is also disclosed.

### TECHNICAL FIELD

The present disclosure relates to devices for removal of material from a patient's body. More particularly, the disclosure relates to such devices that are minimally invasive and have a flexible shaft, as well as related methods of manufacture.

### BACKGROUND

Various devices have been proposed for removing tissue from a desired area within a patient. Such devices often include a tube such as a catheter, lumen, etc., guided to a site of interest and a device of some sort located within the tube to remove tissue from the site of interest.

For example, Hamatura et al. in U.S. Pat. No. 6,554,799, issued Apr. 29, 2003 discloses a biological precision screw pump capable of transferring a sufficient amount of viscous liquid even with a very thin suction and injection pipe. The invention provides a pump capable of minimizing invasion into human bodies by housing a very thin rotor in a cylindrical needle, and positively transferring a liquid based on the mechanical configuration of the rotor. A viscous liquid can be moved by increasing the number of rotations of the rotor and that the pipe diameter can be reduced by twisting up a plurality of thin filaments to obtain a rotor.

Cooke et al. in U.S. Pat. No. 6,926,725 discloses an improvement to a thrombectomy apparatus for breaking up thrombus or other obstructive material in a lumen of a vascular graft or vessel. The wire is operatively connected to a motor for rotation of the wire to enable peaks of the sinuous wire to contact a wall of the lumen to break up the thrombus or other obstructive material. The apparatus comprises a wire being formed of an inner core formed by a plurality of twisted wires and an outer wire wound directly around the inner core. The tightly wound inner/outer core structure enable rotation of the distal of the wire corresponding to rotation at its proximal end as torque is transmitted to its distal end.

U.S. Pat. No. 5,041,082 to Shiber issued Aug. 20, 1991 discloses a mechanical atherectomy system insertable into a patient's artery over a non-rotating, auger shaped flexible guide-wire. A portion of the length of the flexible guide-wire located near the front end is shaped as an auger which is formed by a spaced spiral-wire attached to a core-wire. Once the flexible guide-wire is in place, the flexible rotary catheter and the tubular-blade are advanced to the obstruction site, and continue to be advanced into the obstruction while being rotated over the flexible guide-wire.

U.S. Pat. No. 6,758,851 to Shiber issued Jul. 6, 2004 discloses an apparatus for extracting an obstruction located in a patient's vessel. The apparatus has a flexible-tube with an open distal end that is connected to a negative pressure source. The apparatus further comprises flexible-tube containing a motor rotated conveyor-shaft to which an offset-agitator is connected. The direction of rotation of the conveyor-shaft's spiral is such that as it rotates relative to the flexible tube it conveys the fragments co-operatively with the negative pressure, from the open distal end through the flexible-tube. Shiber discloses that at least a part of the conveyor-shaft and preferably substantially all of its length is a spiral with gaps between its coils to enable the spiral to convey the fragments. The apparatus may be delivered to an obstruction site over a guide-wire

In U.S. Pat. No. 6,926,725, issued Dec. 13, 1988, Hawkins Jr. et al. disclose an apparatus and method for removing a target object from a body passageway. The apparatus comprises a catheter, a spiral wound coil disposed within the catheter and rotatably driven by an air actuated control means and a parachute basket. The spiral wound coil has a cutting tip at its distal end which is housed within the distal tip of the catheter. The target object is fragmented by the cutting action of the tip of the spiral wound coil as it is rotated at high speed within the catheter by the air actuated drive means. Rotation of the spiral wound coil also facilitates transport of the target fragments though the catheter lumen simultaneously with aspiration.

However, in each of the devices above, improvements cold be made in terms of providing a flexible, effective device for removal of tissue. Accordingly, an apparatus for removing tissue from a patient's body that achieved such goals and/or addressed one or more other drawbacks of conventional devices would be welcome.

### SUMMARY

In one broad aspect embodiments of the present disclosure comprise an apparatus for removing tissue from a patient's body, the apparatus comprises: an elongate member defining a lumen; a tissue removal member disposed within the lumen, the tissue removal member comprising a flexible shaft formed from a plurality of strands, and a coil helically disposed around the flexible shaft, the coil being substantially in contact with the shaft along a length of the shaft, the coil forming a plurality of outwardly extending projections, each of the plurality of outwardly extending projections defining a spacing therebetween; wherein rotation of the tissue removal member within the elongate member allows material to be conveyed through the lumen. Various options and modifications are possible.

As a feature of this broad aspect, the flexible shaft comprises a wire rope. As an example of this feature, the wire rope is a 1×7 strand wire rope. As another example of this feature, the wire rope comprises Nitinol. As an alternate example of this feature, the wire rope comprises stainless steel.

As another feature of this broad aspect, the spacing between each pair of the outwardly extending projections is between about 0.001 inches and about 0.1 inches. As an example of this feature, the spacing is about 0.06 inches.

As another feature of this broad aspect, the coil is attached to the flexible shaft at a plurality of locations along the flexible shaft. As an example of this feature the attachment could be via soldering or welding. As an example, the plurality of locations comprises at least four locations along the shaft.

As still another feature of this broad aspect the elongate member forms a bend. As an additional feature, a portion of the tissue removal member in contact with the bend comprises a coating, wherein the coating reduces the friction between the tissue removal member and the elongate member. As an example, the coating comprises a clear PET liner.

As still another feature of this broad aspect, the coil comprises stainless steel. As an alternate feature of this broad aspect, the coil comprises nitinol.

As still another feature of this broad aspect, the elongate member comprises a distal end defining an opening and having a tip, wherein a distal end of the tissue removal member is recessed from the tip and at least a portion of the tissue removal member protrudes beyond the opening to allow access to tissue distal to the opening.

As an additional feature, the apparatus comprises a motorized source of rotational energy operatively connected to the tissue removal member. As an additional feature, the apparatus comprises a handpiece operatively connected to the tissue removal member. As still an additional feature the apparatus further comprises a motorized source of rotational energy operatively connected to the tissue removal member, wherein the handpiece comprises means for engaging and disengaging the motorized source of rotational energy. As still an additional feature the apparatus further comprises a receptacle operatively connected to the elongate member for receiving the tissue removed from the body, the elongate member defining at least one opening for transferring material from the elongate member to the receptacle. As still an additional feature, the receptacle is detachable from the elongate member.

According to other aspects of the disclosure, a method of manufacturing a tissue removal member includes providing a flexible shaft formed from a plurality of strands; and attaching a coil helically around the shaft, the coil being substantially in contact with the shaft along a length of the shaft, the coil forming a plurality of outwardly extending projections, each pair of the plurality of outwardly extending projections defining a spacing therebetween, the coil being attached to the flexible shaft at a plurality of spaced apart locations along the flexible shaft. As above, various options and modifications are possible.

### BRIEF DESCRIPTION OF THE DRAWINGS

In order that the disclosure may be readily understood, embodiments of the disclosure are illustrated by way of examples in the accompanying drawings, in which:

FIG. 1A is a side view of a tissue-removal apparatus in accordance with an embodiment of the present disclosure;

FIG. 1B is a side perspective view of a shaft of a tissue removal member in accordance with an embodiment of the present disclosure;

FIG. 1C is a cross-sectional view of a shaft of a tissue removal member taken along the line 1C-1C in FIG. 1B;

FIGS. 1D-1E are side views of a tissue-removal member in accordance with various embodiments of the present disclosure;

FIGS. 2A-2E are side views of a tissue removal member in accordance with various embodiments of the present disclosure;

FIGS. 3A-3C are side perspective views of an elongate member in accordance with various embodiments of the present disclosure;

FIGS. 4A-4B are top perspective views of the distal end of an elongate member with a tissue removal member disposed therein, in accordance with various embodiments of the present disclosure;

FIG. 4C is a side perspective view of the distal end of an elongate member with a tissue removal member disposed therein, in accordance with an embodiment of the present disclosure;

FIGS. 5A-5B are top perspective views of the distal end of a tissue removal apparatus, in accordance with an alternate embodiment of the present disclosure;

FIG. 5C is a side view of a tissue-removal apparatus in accordance with an embodiment of the present disclosure;

FIGS. 6A-6B are top perspective views of the distal end of a tissue-removal apparatus, in accordance with an alternate embodiment of the present disclosure;

FIG. 7 is an illustration of a method in accordance with an embodiment of the present disclosure;

FIG. 8 is an illustration of a method in accordance with an embodiment of the present disclosure; and

FIG. 9 is an illustration of a method in accordance with an embodiment of the present disclosure.

### DETAILED DESCRIPTION

With specific reference now to the drawings in detail, it is stressed that the particulars shown are by way of example and for purposes of illustrative discussion of certain embodiments of the present disclosure only, and are presented in the cause of providing what is believed to be the most useful and readily understood description of the principles and conceptual aspects of the disclosure. In this regard, no attempt is made to show structural details of the disclosure in more detail than is necessary for a fundamental understanding of the disclosure, the description taken with the drawings making apparent to those skilled in the art how the several forms of the disclosure may be embodied in practice.

Before explaining at least one embodiment of the disclosure in detail, it is to be understood that the disclosure is not limited in its application to the details of construction and the arrangement of the components set forth in the following description or illustrated in the drawings. The disclosure is capable of other embodiments or of being practiced or carried out in various ways. Also, it is to be understood that the phraseology and terminology employed herein is for the purpose of description and should not be regarded as limiting.

As used herein, the term “coring” refers to advancing an elongate member defining a lumen and having an open distal end into a tissue, wherein the advancement results in the incorporation or gathering of at least a portion of the tissue into the lumen of the elongate member.

As used herein, the phrase “operatively connected” is intended to mean “coupled or connected, either directly or indirectly, such that the connected structures are operable to perform a desired function”.

As used herein, the term “conveyance” refers to facilitation of movement of a material from one location to another.

As used herein “blunt tip” refers to a tip that does not have a sharp edge or a point.

In one broad aspect, the present disclosure comprises an apparatus for removal of materials from the body of a patient. In one specific embodiment of the method, the apparatus is used for removal of nucleus pulposus tissue from an intervertebral disc. The apparatus may generally comprise a tissue removal member housed at least partially within an elongate member defining a lumen, for example a sheath. The tissue removal member may be any device that functions to convey tissue from the distal end of the elongate member to a portion exterior to the patient's body. In one embodiment, the tissue removal member is a shaft with projections extending outwardly from the shaft. In one embodiment, the tissue removal member is operatively connected to a motor or other source of rotational energy which provides the motion required to remove the tissue. The elongate member may have an open distal end with a bevel face. Furthermore, the elongate member may have a blunt distal tip that is substantially atraumatic.

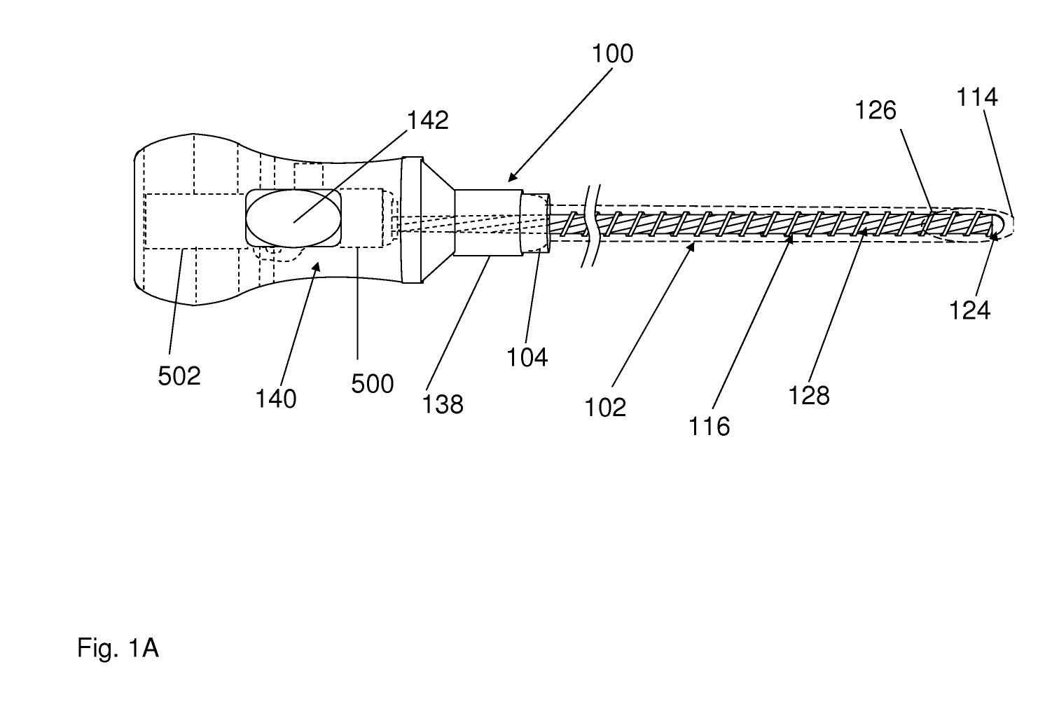

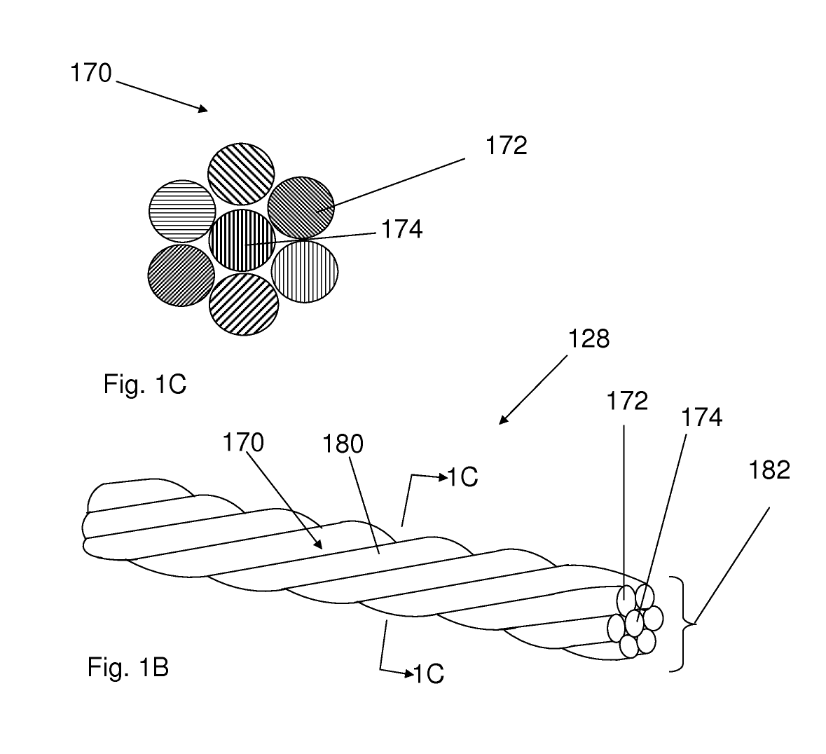

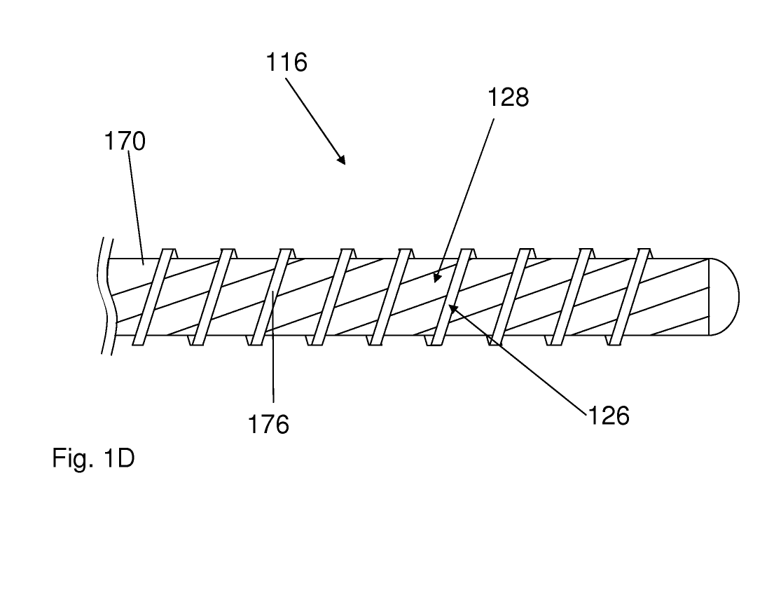

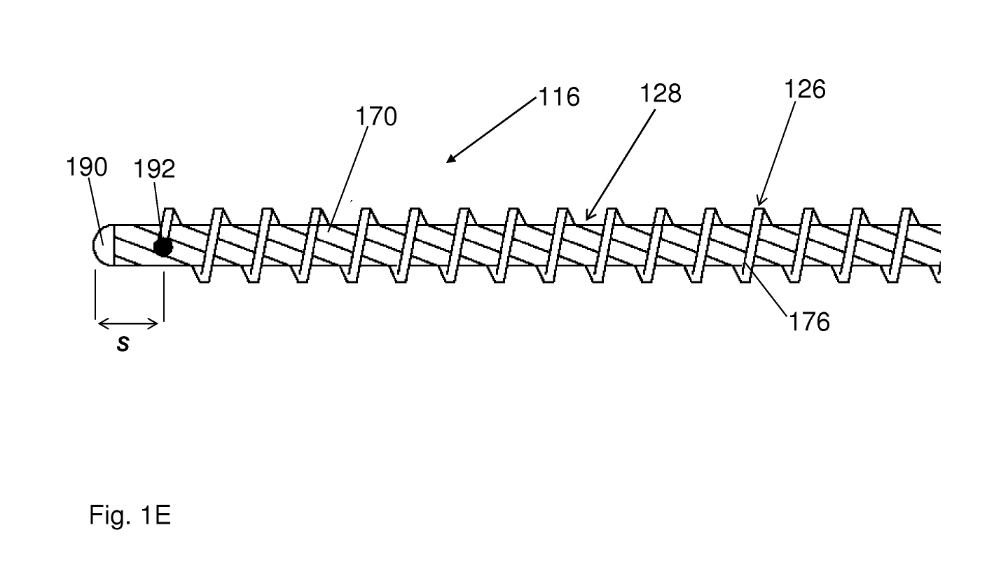

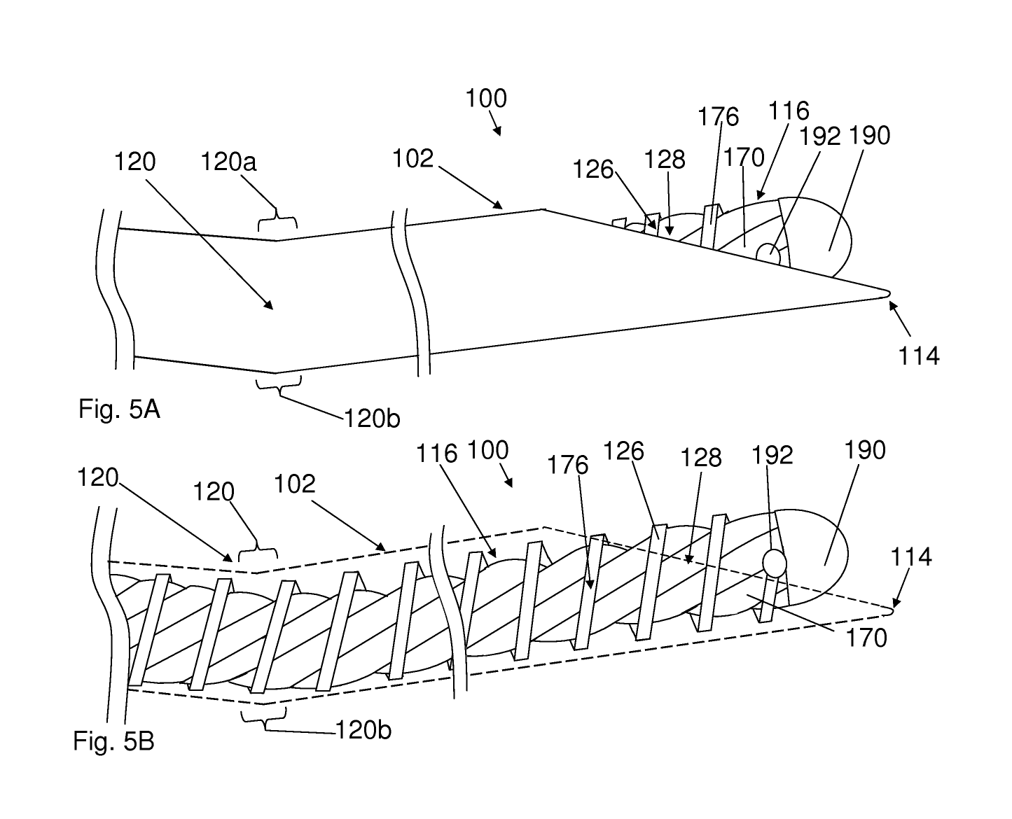

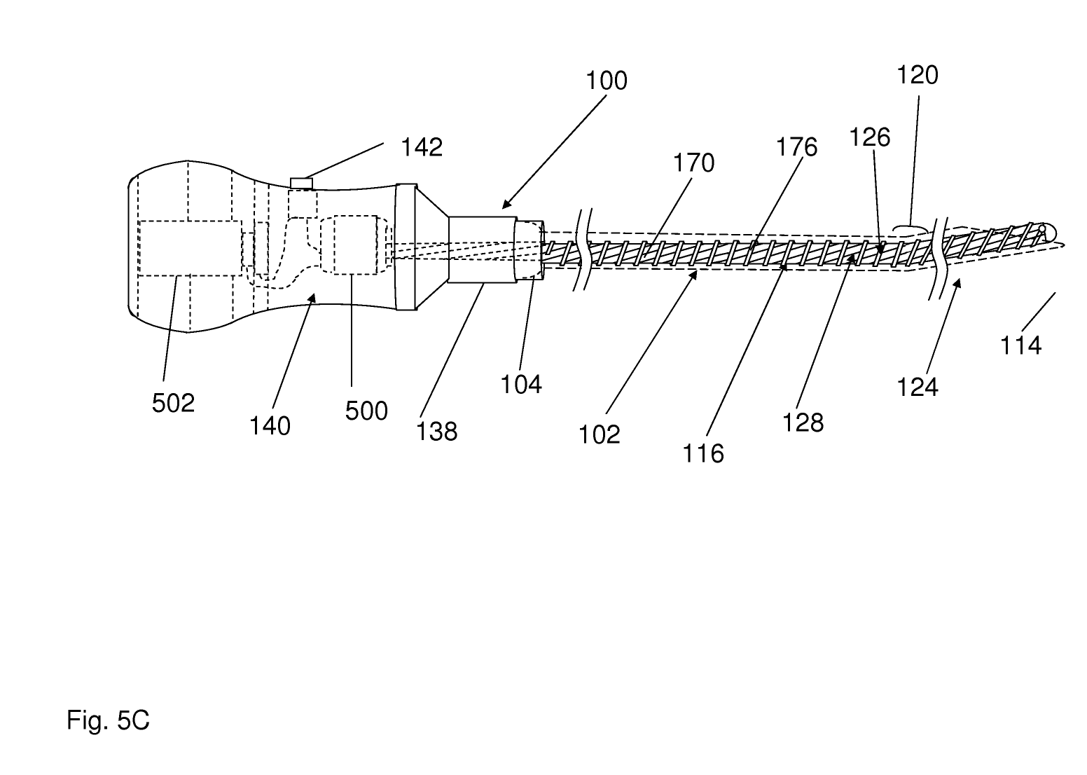

In some embodiments of the present disclosure, for example as shown in FIGS. 1A and 5C, an apparatus 100 is disclosed which comprises an elongate member 102, a tissue removal member 116, a hand piece 140, and a receptacle 138 for collection or visualization of tissue. The tissue removal member 116 is structured to be disposed within elongate member 102. Tissue removal member 116 comprises a proximal portion and distal portion ending in distal end 124. In one embodiment, tissue removal member 116 comprises a shaft 128 having outwardly extending projections 126. In one example, the outwardly extending projections may comprise a helical flighting. In some embodiments the, tissue removal member 116 may be coaxial with elongate member 102; however tissue removal member 116 may be otherwise aligned.

In one embodiment of the present disclosure, as shown in FIGS. 1B, 1C and 1D, the shaft 128 comprises a wire rope 170, comprised of a plurality of strands of intertwined wire 172. The wires 172 comprise a plurality of wires that are twisted together around a central core wire 174 to form a substantially helical structure 180. The helical structure 180 has innately formed flights to aid in the movement of material along the shaft. Thus the wire rope functions to minimally convey, for example viscous material, along the shaft with the aid of indentations in the wire rope 170 as well as the vortex created by the geometry. In one example the wire rope 170 comprises a 1×7 wire rope. In another example the wire rope 170 comprises a 7×3 strand wire rope. The proximity of the tissue removal member 116 (disposed within the lumen of the elongate member) to the inner wall of the elongate member 102 may lead to the generation of heat upon rotation of the tissue removal member 116. The plurality of strands of the wire 172 in the wire rope 170 provide an increased surface area and function to dissipate heat more efficiently when the tissue removal member 116 is rotated within the elongate member 102. The multiple strands of wire 172 together allow the shaft 128 to be supple, but resilient. They allow for faster heat conduction and dissipation which provides a lower thermal gradient.

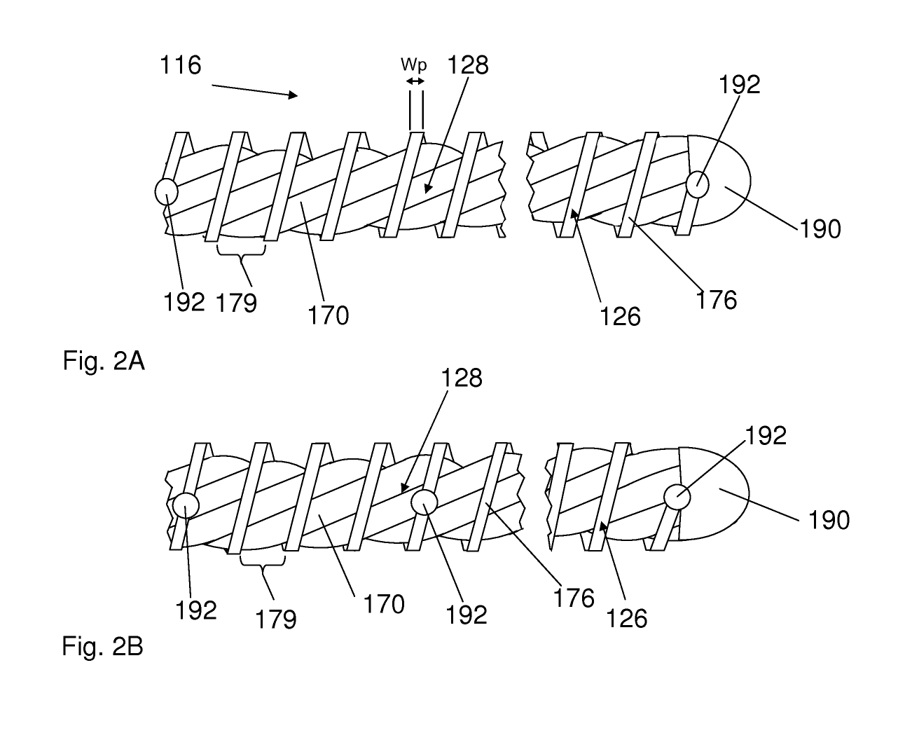

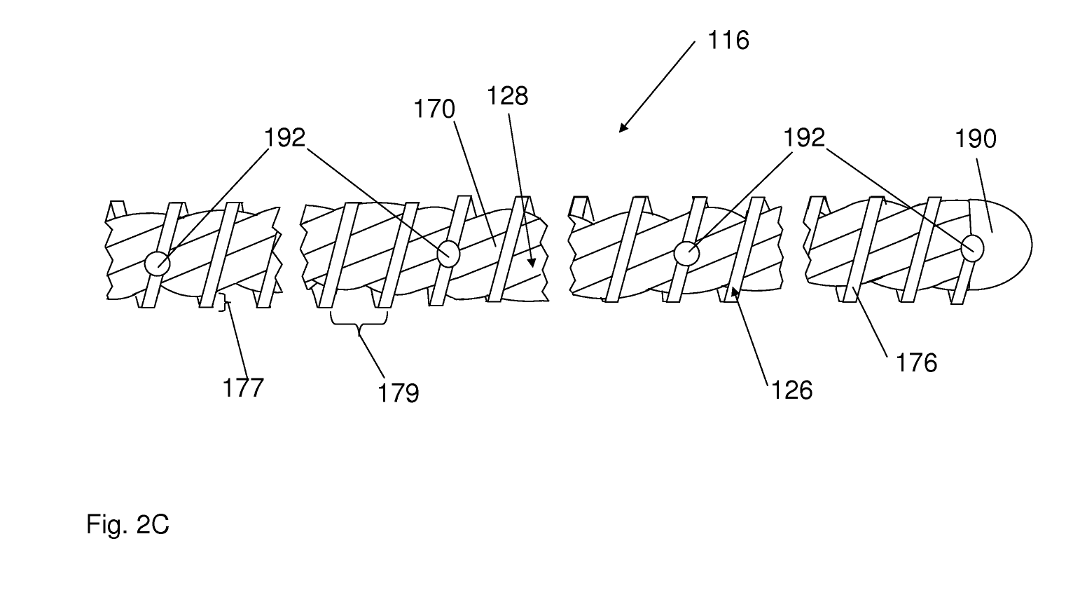

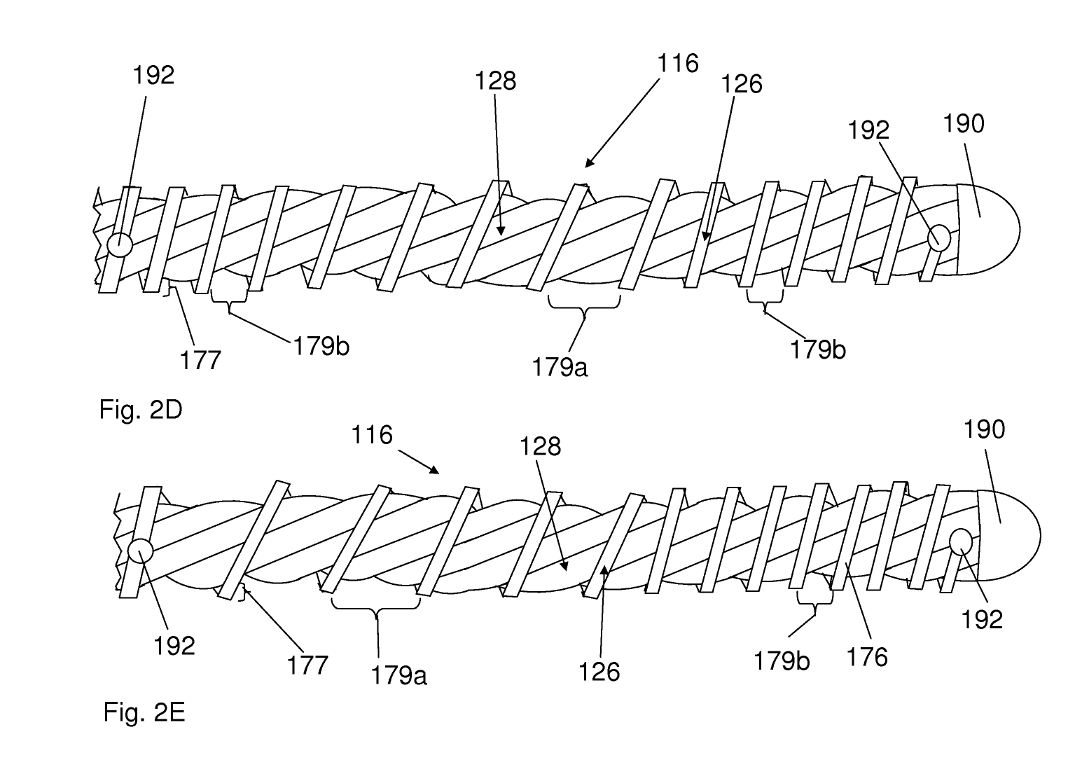

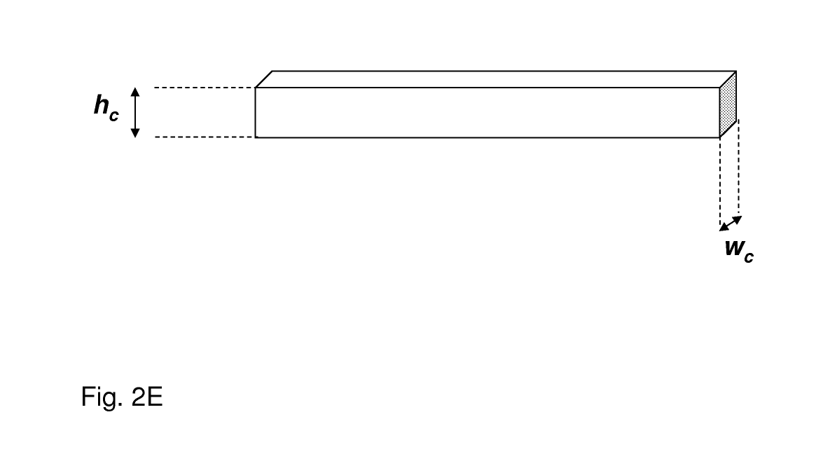

In one embodiment of the present disclosure, as shown in FIGS. 2A, 2B and 2C, the tissue removal member comprises the shaft 128 comprising a wire rope 170 forming a helical structure. In one embodiment the shaft comprises a wire rope 170 that further has a coil of wire 176 helically disposed around the shaft 128. The helical turns of the wire 176 form flights or outwardly extending projections 126. The addition of the outwardly extending projections 126 to the helical structure of the shaft 128 enhances the conveyance of material along the shaft 128 and increases the efficiency of the tissue-removal member 116. In one embodiment the coil of wire 176 has a circular cross-section. In another embodiment of the present disclosure the wire coil 176 has a rectangular cross-section. In one example, the wire coil 176 is a rectangular coil with a cross-section of about 0.004×0.006 inches. In other words, as shown in FIG. 2F, a coil of wire 176 with a height h_{c} of about 0.006 inches and a width W_{c} of about 0.004 inches may be used. In some embodiments the height h_{c} of the coil 176 may be between about 0.004 inches and less than about an inner diameter of the elongate member 102. The width or thickness Wₚ of the outwardly extending projections 126 formed by the coil of wire 176, may generally be equal to width of the coil width W_{c} that is between about 0.003 inches and about 0.025 inches. More specifically the thickness W_{c} of the coil of wire forming the outwardly extending projection 126 is between about 0.005 inches and about 0.010 inches. The coil of wire 176 is helically wound around the shaft such that a spacing 179 exists between adjacent outwardly extending projections 126. In one embodiment of the present disclosure the wire rope 170 is a left lay rope whereas the coil of wire 176 is wrapped in a left hand configuration around the wire rope 170 as illustrated in FIGS. 2A-2E. In other embodiments the wire rope is a right lay rope with a coil of wire 176 wrapped around it in a right hand configuration. In still other embodiments a left lay rope 170 may be used in conjunction with a right hand coil of wire, or similarly a right lay rope may be used in conjunction with a left hand coil of wire.

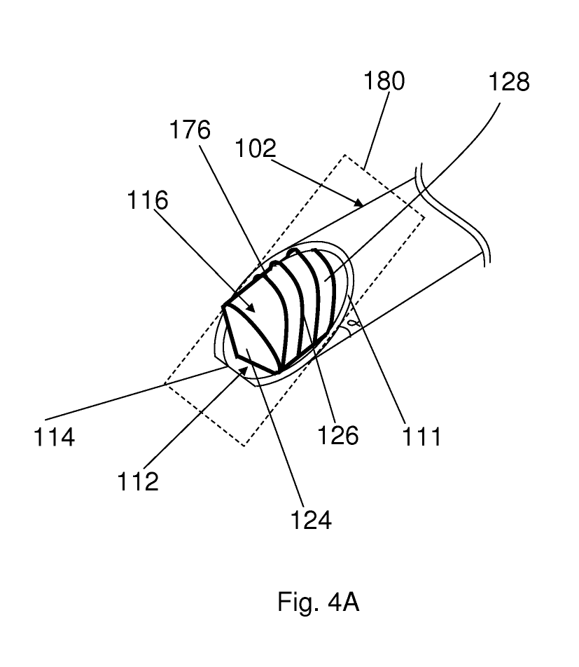

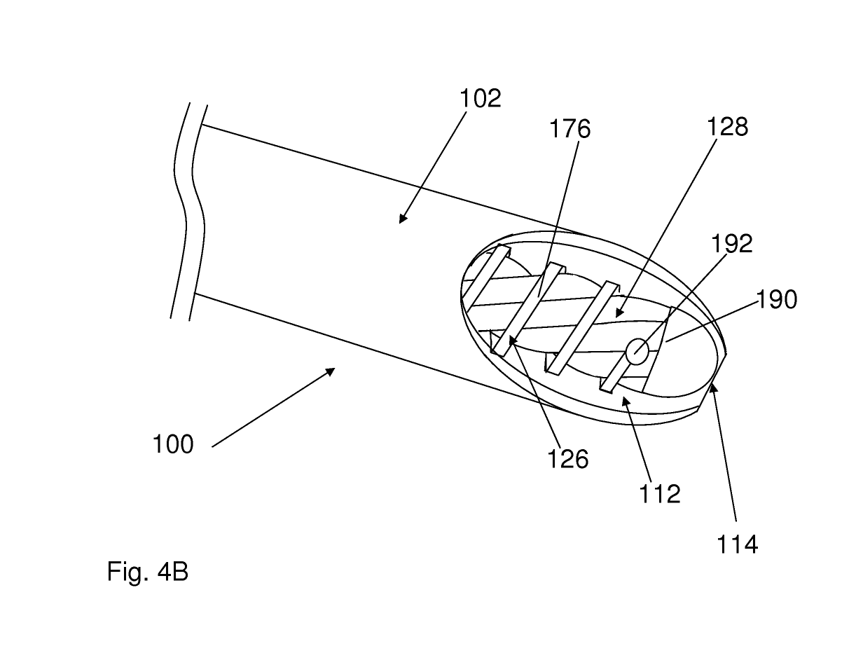

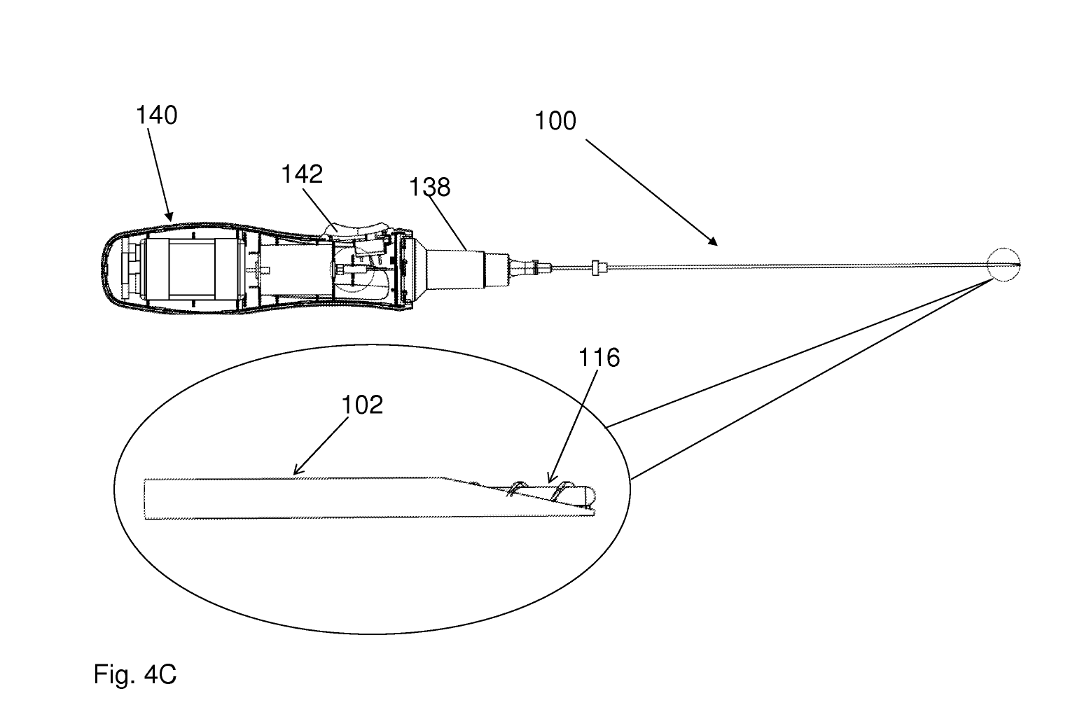

Tissue removal member 116 may generally be between about 6 inches and about 18 inches in length, more specifically between about 8.0 inches and about 15 inches. In one example the length of the tissue removal member is about 9 inches. In one example, the length of the tissue removal member is about 12 inches. In another example the length of the tissue removal member is about 15 inches. The diameter of shaft 128 may generally be between about 0.012 inches and about 0.042 inches, more specifically between about 0.013 inches and about 0.028 inches. The width or thickness Wₚ of the outwardly extending projections 126, as shown in FIG. 2A, may generally be between about 0.003 inches and about 0.025 inches. In one example, the shaft 128 has a diameter of about 0.024 inches. In one example, a 0.004×0.006 inch coil is helically wound around the shaft 128. The distance between adjacent outwardly extending projections 126 or the pitch or spacing 179 is between about 0.03 inches to about 0.06 inches. In other embodiments, the pitch 179 may range from about 0.001 inches to about 0.1 inches. In one example, as shown in FIG. 2C, the distance 177 between the outer diameter of the shaft and the outer diameter of the coil is about 0.012 inches. In one specific embodiment the spacing or pitch 179 is about 0.06 inches. In one specific example, a 12 inch length wire rope 170 is used with an 8 inch length stainless steel coil 176 disposed around it. In another embodiment the spacing or pitch 179 is about 0.04 inches. In one embodiment a distance S between the distal tip of the shaft 128 and the distal end of the stainless steel coil 176 is about 0 inches to about 0.03 inches as shown in FIG. 1E. In one embodiment, the distance S is about 0.03 inches. In other words, the helical flights or outwardly extending projections 126 formed by the coil 176 starts at a distance S of about 0.03 inches from the tip of the auger shaft 128. In one example, the distance S may be about zero inches. In one embodiment, the distance S may be such that it allows about 3 outwardly extending projections 126 to be exposed through the bevel opening 112 when the tissue removal member is inserted into an elongate member as shown by FIGS. 4A-4C.

As shown in FIGS. 2A-2E, the wire coil 176 may be attached to the shaft 128 at a plurality of points along the shaft 128. The wire coil 176 may be attached to the shaft 128 using an adhesive, or alternatively the wire coil may be soldered to the shaft 128. In one embodiment the wire coil 176 is welded at a plurality of locations along the shaft through welds 192. In some embodiments the wire coil 176 is welded to at least two locations along the shaft 128. In one specific example the welds are located at the distal and proximal ends of the wire coil 176 which allows the overall length of the coil to remain constant as shown in FIG. 2A. In other embodiments the wire coil 176 is welded at additional points at defined intervals along the shaft, in additional to the ends of the wire coil 176, as shown in FIG. 2B. In one example the wire coil 176 is attached to the wire rope 170 at its ends as well as at evenly spaced locations along its mid section. In one example the wire coil is attached by welds 192. This may allow limited elastic movement of the wire coil 176 and may limit plastic deformation. In one embodiment the wire coil 176 is welded at least four locations along the shaft as shown by welds 192 in FIG. 2C. As shown in FIG. 2D-2E the coil of wire 176 is free to move laterally between the locations of welds 192. This allows for flexibility or resilience within the coil of wire 176 and can minimize failures. In an embodiment where the tissue removal member 116 is used to convey viscous material, the coil of wire 176 can stretch and deform as material is moved along the tissue removal member 116. This may reduce breakage by reducing the stress placed on weld points 192. At the distal tip of the auger the wire rope 170 is welded at the distal tip to form a ball weld 190. This may reduce fraying of the wire rope at the tip and can allow tissue to glide over the tip of the tissue removal member 116 and travel along the tissue removal member 116. In one example the ball weld 190 comprises a half sphere at the distal tip of the tissue removal member 116. In an alternate embodiment silver solder may be used at the distal tip to prevent fraying. In some embodiments the wire coil 176 may be welded to the shaft 128 using a weld 192 at the ball weld 190. In one example, the wire coil 176 is formed from a medical grade stainless steel, such as stainless steel 304 or stainless steel 316. In an alternate embodiment the wire coil 176 may be constructed from Mp35N metal with an iron-base. In an alternate embodiment the wire coil 176 may be constructed from Nitinol.

In accordance with one embodiment of the present disclosure, a tissue removal apparatus is disclosed which comprises a tissue removal member 116 disposed within an elongate member 102 as illustrated in FIGS. 4A, 4B and 4C. The tissue removal member 116 may be positioned within the elongate member 102 such that at least some of the outwardly extending projections 126 of the tissue removal member 116 are protruding from the opening 112. In some embodiments about 2 to about 5 of the outwardly extending projections 126 may be exposed to the opening 112. In some embodiments as shown in FIG. 4B about 3 to 4 turns of the helical wire 176 forming the outwardly extending projection 126 may be exposed to the opening 112. In one example, as shown in FIG. 4C, about 3 turns of the helical wire 176 are exposed. In some embodiments less than about 2 turns or greater than about 5 turns may be exposed.

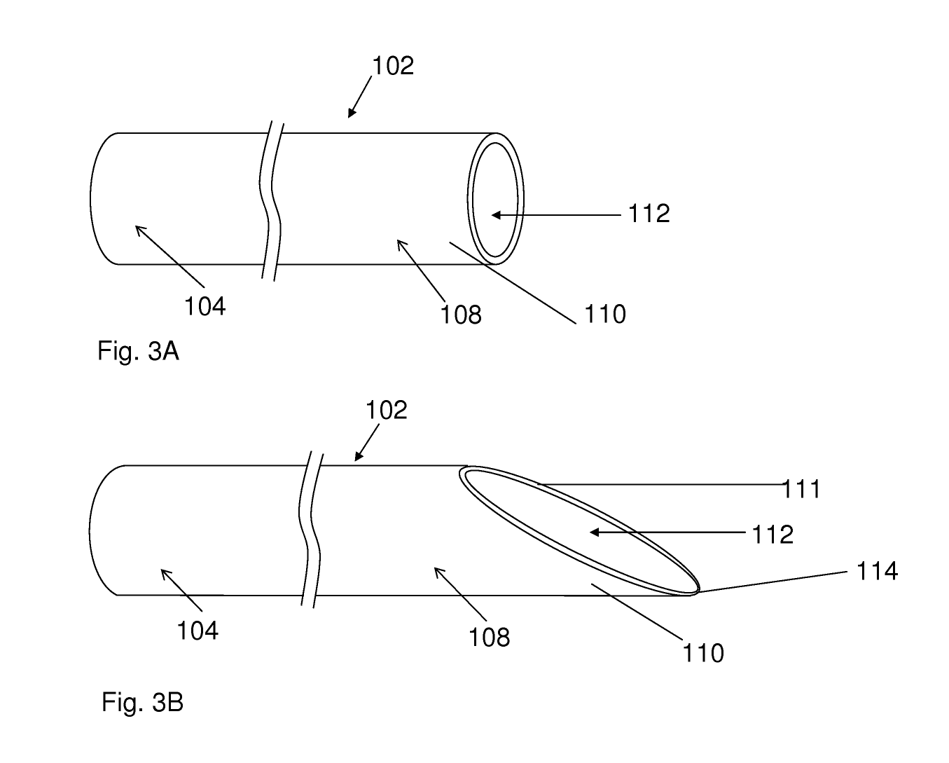

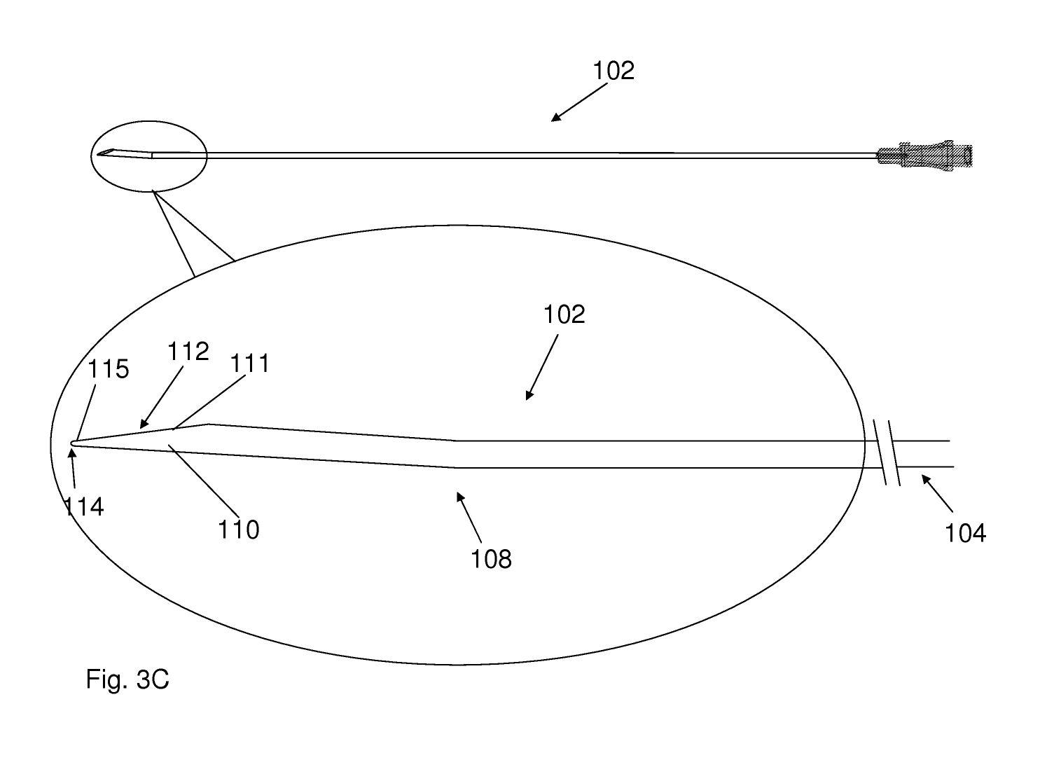

In the general embodiment shown in FIG. 3A, an elongate member 102 is disclosed that comprises a proximal portion 104 and distal portion 108 ending in an open distal end 110. The open distal end 110 defines an opening 112. In one specific embodiment, the cross-sectional shape of the elongate member 102 is substantially circular; however alternate embodiments are possible, wherein the shape may be ovoid, square, or rectangular and elongate member 102 is not limited in this regard. In the illustrated embodiment, the opening 112 is substantially perpendicular to the longitudinal axis of the shaft.

Elongate member 102 may be manufactured from a number of different materials. These include, but are not limited to, stainless steels, shape-memory materials such as nickel titanium alloys, polyesters, polyethylenes, polyurethanes, polyimides, nylons, copolymers thereof, and medical grade plastics. In one specific embodiment, elongate member 102 is made from a clear, transparent or translucent plastic or other material. This embodiment may allow the user to visualize the contents of elongate member 102 to ensure that the elongate member (or any other device disposed within the elongate member) is operating properly. This may allow for visibility in order to see if material (for e.g. tissue) is being conveyed or if there is a blockage. In one specific example, the elongate member 102 may be made from stainless steel. In one embodiment the elongate member 102 may not be bent. In one specific example of this embodiment, the elongate member is made from Nitinol. Nitinol has elastic properties which may prevent the elongate member 102 from being permanently deformed or bent when force is applied.

FIG. 3B illustrates a specific embodiment of the present disclosure where the open distal end 110 comprises a bevel face 111 defining an opening 112. The plane in which the opening or aperture 112 is defined may not be perpendicular to the longitudinal axis of elongate member. The bevel face 111 is cut at an angle with respect to the longitudinal axis of the elongate member 102. In one example the cut is performed using a laser. In an alternate example the elongate member 102 is cut using Electrical Discharge Machining (EDM). In alternate embodiments any other means of cutting may be used to form the bevel face 111. In some embodiments as shown in FIG. 3B, the tip of the bevel face 111 has been altered such that it is no longer sharp and has a blunt tip 114. In one example the blunt tip 114 is formed by grit-blasting electrochemical polishing, shaving, sanding, using a laser to cut it into shape or using any other means.

In one specific embodiment, elongate member 102 is sized to be percutaneously directed to an interior tissue of the body. The length of elongate member 102 is generally between about 5 inches to about 12 inches; however it may be otherwise sized to reach any target tissue within the body. In one specific example, the length of the elongate member is about 5.2 inches. In another example the length is about 8.2 inches. In a still further example the length is about 11.2 inches. The elongate member 102 may comprise a hypo-tube of between about 14 Gauge to about 20 Gauge. In some embodiments the elongate member 102 may comprise a hypotube of less than about 14 Gauge. In other words, the outer diameter 166 of the elongate member may be between about 0.030 inches to about 0.090 inches; however it may be otherwise sized to fit within the space defined by the target tissue. In some embodiments the outer diameter 166 may be greater than 0.090 inches. In one specific example, the elongate member 102 may comprise a 19 Gauge thin wall hypotube. The inner diameter 164 may be about 0.033 inches and the outer diameter 166 may be about 0.042 inches and the wall thickness T_{w} is about 0.004 inches. In another example, the elongate member 102 may comprise an 18 Gauge thin wall hypotube. The inner diameter 164 may be about 0.042 inches and the outer diameter 166 may be about 0.050 inches and the wall thickness T_{w} of the elongate member 102 may be about 0.004 inches. In other embodiments, the wall thickness T_{w} may be between about 0.0035 inches to about 0.01 inches.

In one embodiment, elongate member 102 may be bent or curved as shown in FIG. 3C and FIGS. 5A, 5B and 5C. This may allow for easier access to a target site. The bend or curve 120 may be applied by the user prior to or during the procedure, or may be applied during manufacture. For example, if the target tissue is the nucleus pulposus of an intervertebral disc, the curve or bend 120 may be at such an angle that the posterior portion of the nucleus pulposus may be reached with the device while allowing for an approach that reduces risk of damage to the spinal canal. The bend may also facilitate a larger coring radius for more tissue removal. In one embodiment, the tissue removal apparatus 100 may be used within the intervertebral disc. And the angle of the bend, or in other words the angle of curvature of the elongate member 102 may be between about 0° to about 8.6° with respect to the longitudinal axis of the elongate member 102. More specifically, the angle of curvature may be between about 6.6° and about 8.6°. In one example, the bend has an angle of about 7.6°. In other embodiments, any suitable angle of curvature or bend may be used. Furthermore, the curve or bend 120 may be located at various points along the length of the elongate member. In some embodiments, the bevel face 111 may face upwards with the bend 120. In other words the bevel face 111 may be positioned on the same side as the upper side 120a of the bend 120. Whereas, in other embodiments the bevel face 111 may be positioned at the opposite side or the lower side 120b of the bend 120. In still other embodiments the bevel face may be positioned in other orientations with respect to the bend 120. A tissue removal member 116 may be disposed within the elongate member 102. The tissue removal member 116 comprising a flexible shaft 128 formed from the wire rope 170 allows the tissue removal member 116 to conform to the shape of the elongate member 102 as shown in FIG. 5B, specifically at the bend 120, reducing the friction between the elongate member 102 and the tissue removal member 116. Furthermore, the wire rope 170 forming the shaft 128 may reduce mechanical breaking during bending and rotation. This may help reduce the risk of the tissue-removal member from breaking off inside a patient during use, leaving metallic pieces behind or creating thermal necrosis. Additionally the wire rope 170 of the shaft 128 helps to transfer heat away from the bend 120 where the friction would be the greatest.

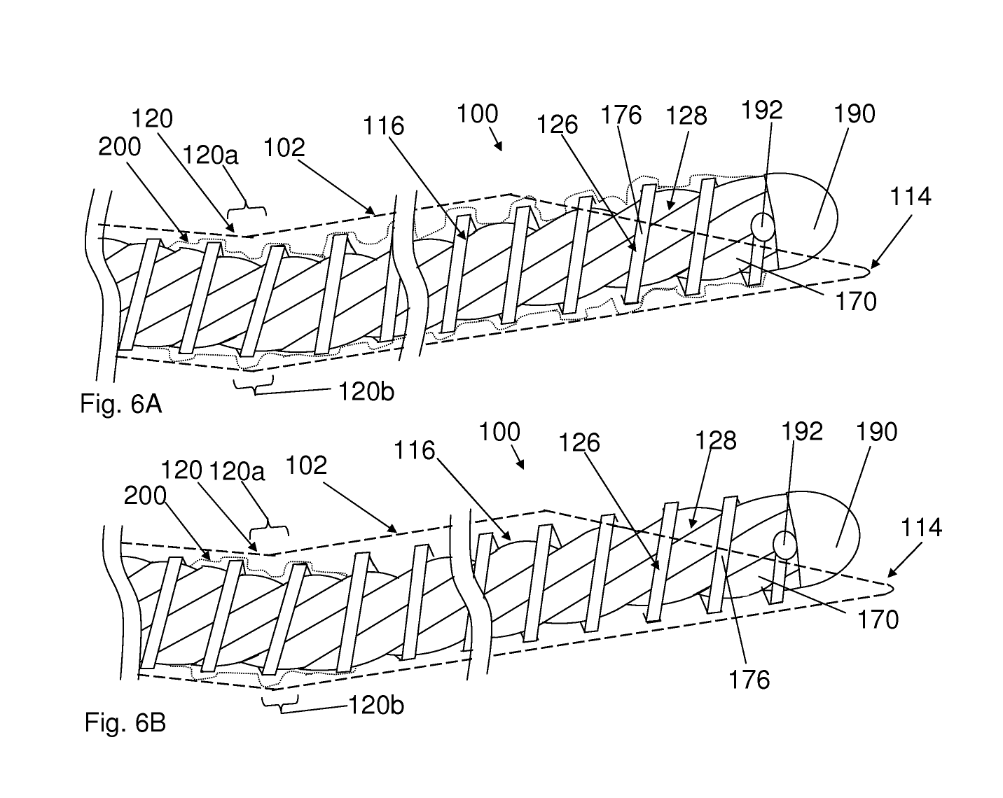

In some embodiments a coating 200 may be applied to the tissue removal member 116 encasing the coil of wire 176 and the wire rope 170 to help reduce the friction as illustrated in FIGS. 6A-6B. In one embodiment the coating 200 may be a polymer coating. In one example, the coating 200 is a Polyethylene terephthalate (PET) liner. In one specific example, the coating 200 is a clear PET liner that has been applied to the tissue-removal member 116 using a heat-shrink process. In some embodiment the coating 200 may be applied to the tissue removal member 116 substantially along its length as shown in FIG. 6A. In other embodiments the coating 200 may be applied along a portion of the tissue removal member 116. In one example the coating 200 may be applied to the tissue removal member 116 just at the bend 120 as shown in FIG. 6B. In other embodiments, the coating 200 may be applied only at specific locations along the tissue removal member for example around the welds 192. The coating 200 may help prevent particulate generation by reducing the direct contact or interaction of the tissue removal member 116 with the inner lumen of the elongate member 102. The liner may help especially in the instance where particulate generation may be of concern where there is metal-to-metal interaction between the tissue removal member 116 and elongate member 102. In one embodiment, the coating 200 may have a thickness of about 0.00025 inches, to about 0.001 inches. In one specific embodiment, the coating 200 has a thickness of about 0.0005 inches. In one example, the coating 200 is a heat shrink liner. In some embodiments the coating 200 may comprise a polytetrafluoroethylene PTFE liner. In one example, the coating 200 may be an electronic deposition PTFE. In some embodiments, the coating 200 may be applied to the inner lumen of the elongate member 102.

As previously mentioned the apparatus 100 shown in FIGS. 1A and 5C may comprise a receptacle or collection chamber, 138 for housing and/or viewing any material, for e.g. tissue that may be removed from the body. The collection chamber 138 is operatively connected to the elongate member 102 for receiving the material that may be removed from the body. The elongate member 102 defines at least one opening for transferring material from the elongate member 102 to the collection chamber 138. In some embodiments, collection chamber may be structured to be coupled to handpiece 140. In yet another embodiment, collection chamber 138 may be located within handpiece 140. In some embodiments, collection chamber 138 may be structured to allow the user to visualize and measure the amount of material such as tissue in the chamber and may be detachable. In one embodiment, the handpiece 140 may be structured to house a battery 502, a motor 500, and electrical connections therebetween. The motor 500 may be operatively connected to the shaft of the tissue removal member 116. In addition, a button or switch 142 may be located on handpiece 140.

In some embodiments, tissue removal member 116 may be operatively connected to a source of motorized rotational energy, for example a motor 500, to allow for rotation of tissue removal member 116. In one specific embodiment, motor 500 may be connected to battery 502. When a switch 142 is engaged, motor 500 may cause shaft 128 of tissue removal member 116 to rotate, thereby rotating outwardly extending projections 126 and conveying tissue from the distal portion 122 of tissue removal member 116 to proximal portion 118. In one embodiment of the present disclosure where a left lay rope 170 with a left hand coil of wire is used 176, the motor 500 and hence the tissue removal member 116 is rotated clockwise. In other embodiments where a right lay rope with a right hand coil of wire is used, the motor direction may be reversed and it may be rotated counterclockwise. In still other embodiments, the motor may be rotated counterclockwise or clockwise and the tissue removal member may have varying combinations of rope lay and coil wrapping configurations. An elongate member 102 is comprises a hub 106 that mates with the distal portion of the handle. In one embodiment the hub 106 of the elongate member 102 comprises luer threads that engage with a luer on the distal portion of the handle. Generally, the rotation of the tissue removal member 116 within the elongate member 102 may generate heat. This may increase the temperature of the tissue removal member 116 and/or the elongate member 102 such that it is above body temperature. In one specific embodiment, the elongate member 102 is fabricated from Nitinol. The thermal properties of Nitinol allow for greater heat dissipation which may help to minimize any changes in temperature resulting from heat generation due to rotation of the tissue removal member 116 within the elongate member 102.

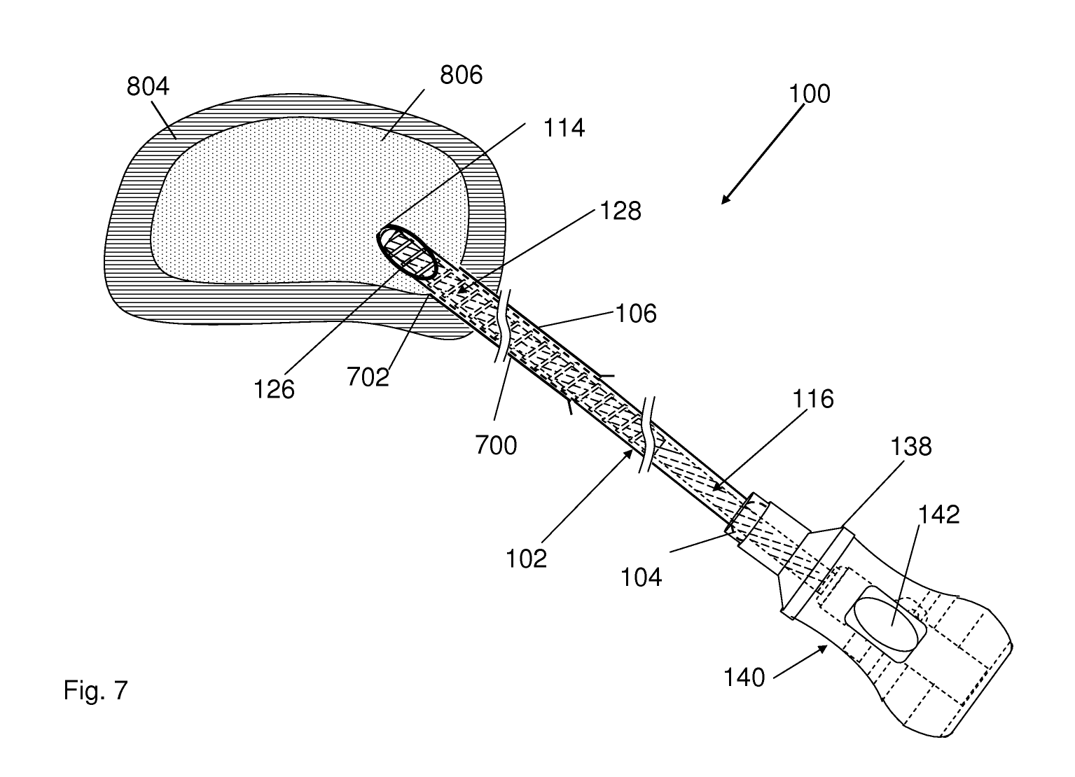

In some embodiments, as shown in FIGS. 7-9, apparatus 100 may comprise an introducer apparatus that will aid in introducing elongate member 102 into the target tissue. The introducer apparatus may include a hollow elongate introducer or cannula 700 and an obturator. Cannula 700 may be substantially stiff or rigid, such that it may assist in piercing skin or other body tissues, or such that it may provide support for apparatus 100. The obturator may be structured to cooperatively engage with cannula 700. In some embodiments the distal tip of the obturator may be sharp and may be conical, beveled, or more specifically, tri-beveled or trochar tipped. Further details regarding various embodiments of the apparatus are provided in U.S. application Ser. Nos. 11/128,342, 11/368,491, 11/368,505, 11/368,506, 11/368,508, 11/368,509, 11/368,510, 11/368,475 and 11/368,513, incorporated herein by reference.

In one broad aspect, the disclosure comprises methods for removal of material from a body. The methods described herein may be used to remove various types of materials from a patient's body. Examples of such materials include, but are not limited to, tissue of an intervertebral disc (for example, the nucleus pulposus), tumor tissue (including, but not limited to, material from breast, colon, stomach, or liver tumors), bone tissue (for example, bone marrow), cyst material, adipose tissue, eye material, cartilage, or atherosclerotic material. In one embodiment, the method of the present disclosure may be practiced using apparatus 100, including tissue removal member 116 disposed within elongate member 102, as described hereinabove. In one example, the apparatus 100 may be used as a disc-decompression device. In one embodiment of the present disclosure an introducer apparatus, as illustrated in FIG. 7, comprising a cannula 700 and an obturator, is advanced until distal end 702 of cannula 700 is positioned at the target site which is the boundary between the annulus fibrosus 804 and the nucleus pulposus 806. The obturator is then withdrawn proximally from cannula 700, leaving a distal end 702 of cannula 700 at the leading annulus wall of the intervertebral disc. The elongate member 102 may then be inserted through the lumen of cannula 700 and advanced until the blunt distal tip 114 of the elongate member 102 is located distal to distal end 702 of cannula 700.

Tissue may then be removed using the electrosurgical device by engaging the motor and activating the tissue removal member in order to remove tissue. The elongate member may be advanced through the target site and material can be conveyed away from the target site. After the desired volume of tissue has been removed the apparatus 100 may be removed from the body, as discussed further below. In one embodiment, tissue removal member 116 is coupled to motor 500. Upon engagement of motor 500, tissue removal member 116 rotates about its longitudinal axis. Outwardly extending projections 126, described hereinabove, will engage the tissue within elongate member 102, and convey the tissue toward proximal end of tissue removal member 116. In one specific embodiment, proximal portion of tissue removal member 116 is operatively connected to collection chamber 138 as described hereinabove. As described above and as shown in FIG. 2D-2E the coil of wire 176 forming the outwardly extending projections 126 is free to move laterally between the points of welds 192. This is advantageous in conveying material of the nucleus pulposus which is dense and viscous and may place additional stress on the tissue removal member 116. The tissue medium within the nucleus pulposus of the intervertebral disc has a viscous consistency containing both fluid and solid materials. To account for the variance in viscosity and solid particulates that will travel between the tissue removal member 116 and the elongate member 102, the tissue removal member 116 allows coil of wire forming the outwardly extending projections 126 to move laterally. Thus the outwardly extending projections 126 may move laterally altering the spacing 179 between them. Welding the wires at spaced apart locations along the shaft 128 allows for flexibility or resilience within the coil of wire 176 as the wire can stretch and deform as viscous material is moved along the tissue removal member 116. The spacing 179 between the outwardly extending projections 126 may change as shown in FIGS. 2D and 2E. This may help minimize failures caused by breakage by reducing the stress placed on weld points 192. As the material is conveyed by the coils of wire 176, looking at a section of the shaft between welds 192, the spacing 179b between some outwardly extending projections 126 is altered such that it is less than the standard spacing 179, whereas spacing 179a between some outwardly extending projections 126 increases. Thus the spacing 179 may become variable as the outwardly extending projections 126 are able to move with the viscous fluid. This movement aids in minimizing stress failures resulting from the wire coil 176 detaching from the shaft 128.

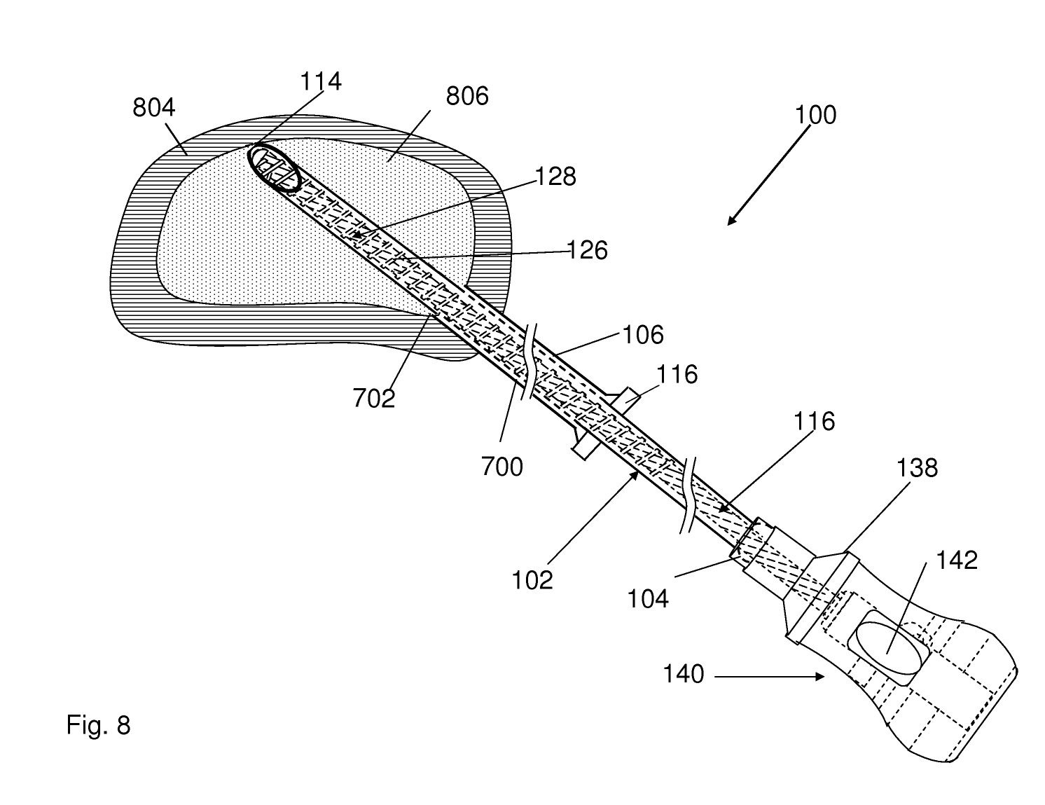

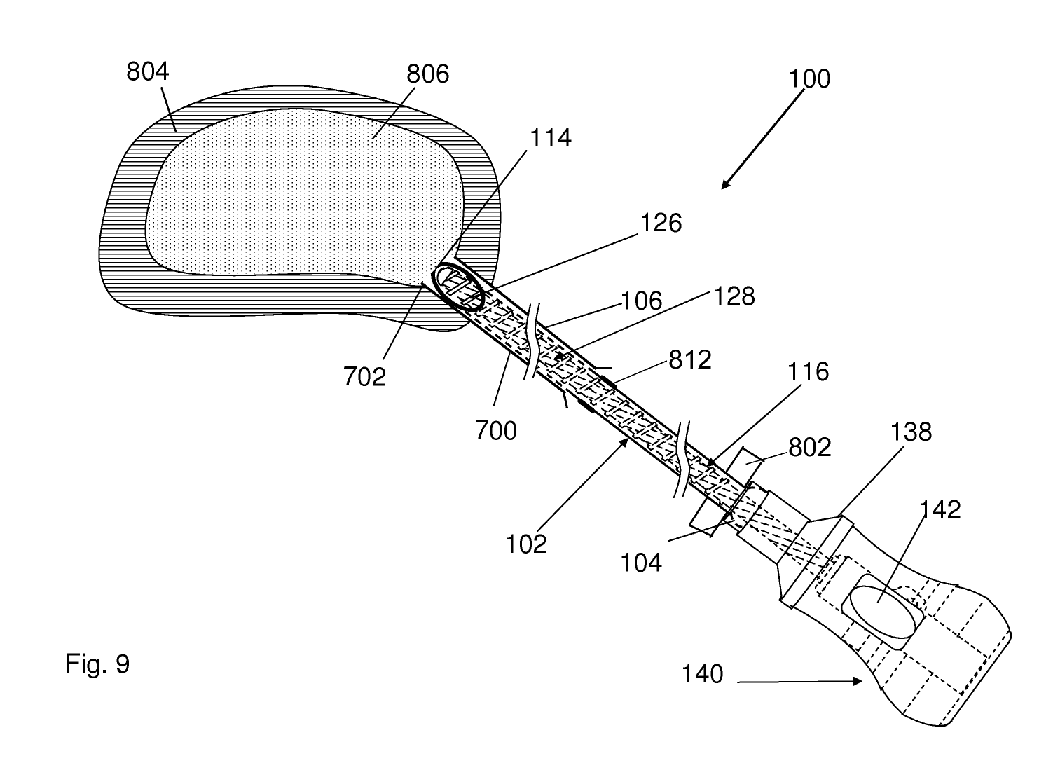

In some embodiments, after the tissue removal apparatus 100 has been positioned at the leading annulus wall, the user may then advance elongate member 102 through nucleus pulposus 806, without activating tissue removal member 116, until the blunt distal tip 114 of elongate member 102 contacts annulus fibrosis 804 on the anterior side or portion of the disc, as shown in FIG. 8. This point of contact may be referred to the as the “anterior annulus inner wall” of the intervertebral disc. The boundary between nucleus pulposus 806 and annulus fibrosis 804 may be located, for example, by tactile sensation, as the annulus fibrosis 804 is generally stiffer than the nucleus pulposus 806, or by using a contrast solution and performing the method under fluoroscopy, as described hereinabove. Once the distal tip end 114 has been positioned at the anterior annulus inner wall, the user may place a marker or depth stopper 802 on the distal most portion of elongate member 102 that is proximal to the proximal end of cannula 700. After the depth stopper 802 has been placed, the user may then retract the blunt distal tip 114 of elongate member 102 proximally to distal end 702 of cannula 700. In one embodiment, proximal portion 104 of elongate member 102 may comprise a marking 812 located such that when the marking is aligned with the proximal end of cannula 700, it indicates that the distal ends of cannula 700 and elongate member 102 are aligned as shown in FIG. 9. Thus, when elongate member 102 is withdrawn proximally through the disc, the user will know to stop retracting elongate member 102 when marking 812 is aligned with the proximal end of cannula 700. The user may then engage tissue removal member 116, and begin the coring and conveyance procedure. As shown in FIG. 8, the user may stop advancing apparatus 100 through the disc as a depth-stopper 802 approaches the proximal end of cannula 700. In an alternate embodiment a marker may be used instead of the depth-stopper 802. The user may then withdraw apparatus and repeat the coring and conveying step. The use of a marker or depth stopper 802 may help to ensure that the distal tip 114 of elongate member 102 does not contact annulus fibrosis 804. This may be especially advantageous in the case of a severely damaged disc that may be severely affected by damage to annulus fibrosis 804. In accordance with an embodiment of the present disclosure, the tissue removal member 116 allows to limit heat generation at the distal tip 114 as well as at the bend 120 of the elongate member 102, as shown in FIGS. 5A and 5B. The shaft 128 of the tissue removal member 116 is flexible, allowing the tissue removal member 116 to conform to the shape of the elongate member 102. This minimizes the heat generation at the bend 120. Additionally the multiple strands of wire 172 that function to dissipate heat more efficiently, also functioning to minimize heat generation. Thus any negative effects that may result from excessive heating of tissue may be mitigated. Thus, the tissue removal member 116 of the present disclosure may prevent denaturing of tissue adjacent to the tissue removal apparatus 100 during use.

In one broad aspect embodiments of the present disclosure comprise an apparatus for removing tissue from a patient's body, the apparatus comprises: an elongate member defining a lumen; a tissue removal member disposed within the lumen, the tissue removal member comprising a flexible shaft formed from a plurality of strands, and a coil helically disposed around the flexible shaft, the coil being substantially in contact with the shaft along a length of the shaft, the coil forming a plurality of outwardly extending projections, each of the plurality of outwardly extending projections defining a spacing therebetween; wherein rotation of the tissue removal member within the elongate member allows material to be conveyed through the lumen.

As a feature of this broad aspect, the flexible shaft comprises a wire rope. As an example of this feature, the wire rope is a 1×7 strand wire rope.

As another feature of this broad aspect, the spacing between each pair of the outwardly extending projections is between about 0.001 inches and about 0.1 inches. As an example of this feature, the spacing is about 0.06 inches.

As another feature of this broad aspect, the coil is attached to the flexible shaft at a plurality of locations along the flexible shaft. As an example of this feature the attachment could be via soldering or welding. As an example, the plurality of locations comprises at least four locations along the shaft.

As still another feature of this broad aspect the elongate member forms a bend. As an additional feature, a portion of the tissue removal member in contact with the bend comprises a coating, wherein the coating reduces the friction between the tissue removal member and the elongate member. As an example, the coating comprises a clear PET liner.

As still another feature of this broad aspect, the coil comprises stainless steel. As an alternate feature of this broad aspect, the coil comprises Nitinol.

As still another feature of this broad aspect, the elongate member comprises a distal end defining an opening and having a tip, wherein a distal end of the tissue removal member is recessed from the tip and at least a portion of the tissue removal member protrudes beyond the opening to allow access to tissue distal to the opening.

As an additional feature, the apparatus comprises a motorized source of rotational energy operatively connected to the tissue removal member. As an additional feature, the apparatus comprises a handpiece operatively connected to the tissue removal member. As still an additional feature the apparatus further comprises a motorized source of rotational energy operatively connected to the tissue removal member, wherein the handpiece comprises means for engaging and disengaging the motorized source of rotational energy. As still an additional feature the apparatus further comprises a receptacle operatively connected to the elongate member for receiving the tissue removed from the body, the elongate member defining at least one opening for transferring material from the elongate member to the receptacle. As still an additional feature, the receptacle is detachable from the elongate member.

The embodiments of the disclosure described above are intended to be exemplary only. The scope of the disclosure is therefore intended to be limited solely by the scope of the appended claims.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure.

We claim:

1. An apparatus for removing tissue from a patient's body comprising:
an elongate member defining a lumen; and
a tissue removal member disposed within the lumen, said tissue removal member comprising a flexible shaft formed from a plurality of strands, and a coil helically disposed around said shaft, said coil being substantially in contact with said shaft along a length of said shaft, said coil forming a plurality of outwardly extending projections, each pair of said plurality of outwardly extending projections defining a spacing therebetween;
wherein rotation of said tissue removal member within said elongate member allows material to be conveyed through said lumen.

2. The apparatus of claim 1 wherein said flexible shaft comprises a wire rope.

3. The apparatus of claim 2, wherein said wire rope is a 1×7 strand wire rope.

4. The apparatus of claim 1, wherein said spacing between each pair of said outwardly extending projections is between about 0.001 inches and about 0.1 inches.

5. The apparatus of claim 4, wherein said spacing is about 0.06 inches.

6. The apparatus of claim 1, wherein said coil is attached to said flexible shaft at a plurality of locations along said flexible shaft.

7. The apparatus of claim 6, wherein said coil is attached to the shaft via soldering.

8. The apparatus of claim 6, wherein said coil is attached to the shaft via welding.

9. The apparatus of claim 6, wherein said plurality of locations comprises at least four locations along said shaft.

10. The apparatus of claim 9, wherein said coil is configured to be able to move laterally between each of said at least four locations.

11. The apparatus of claim 1, wherein said elongate member forms a bend.

12. The apparatus of claim 10, wherein a portion of said tissue removal member in contact with said bend comprises a coating, wherein said coating reduces the friction between said tissue removal member and said elongate member.

13. The apparatus of claim 11, wherein said coating comprises a clear PET liner.

14. The apparatus of claim 2, wherein said wire rope comprises Nitinol.

15. The apparatus of claim 2, wherein said wire rope comprises stainless steel.

16. The apparatus of claim 1, wherein said coil comprises stainless steel.

17. The apparatus of claim 1, wherein said coil comprises nitinol.

18. The apparatus of claim 1, wherein said elongate member comprises a distal end defining an opening and having a tip, wherein a distal end of said tissue removal member is recessed from said tip and at least a portion of the tissue removal member protrudes beyond said opening to allow access to tissue distal to said opening.

19. The apparatus of claim 18 further comprising a motorized source of rotational energy operatively connected to said tissue removal member.

20. The apparatus of claim 19, further comprising a handpiece operatively connected to said tissue removal member.

21. The apparatus of claim 20, further comprising a motorized source of rotational energy operatively connected to said tissue removal member, wherein said handpiece comprises means for engaging and disengaging said motorized source of rotational energy.

22. The apparatus of claim 21, further comprising a receptacle operatively connected to the elongate member for receiving the tissue removed from the body, said elongate member defining at least one opening for transferring material from said elongate member to said receptacle.

23. The apparatus of claim 22, wherein said receptacle is detachable from said elongate member.

24. A method of manufacturing a tissue removal member comprising:
providing a flexible shaft formed from a plurality of strands; and
attaching a coil helically around said shaft, said coil being substantially in contact with said shaft along a length of said shaft, said coil forming a plurality of outwardly extending projections, each pair of said plurality of outwardly extending projections defining a spacing therebetween, the coil being attached to said flexible shaft at a plurality of spaced apart locations along said flexible shaft.

25. The method of claim 24, wherein said coil is attached to the shaft via soldering.

26. The method of claim 24, wherein said coil is attached to the shaft via welding.

27. The method of claim 24, wherein said plurality of locations comprises at least four locations along said shaft.

28. The method of claim 27, wherein said coil is configured to be able to move laterally between each of said at least four locations.

29. The method of claim 24 wherein said flexible shaft comprises a wire rope.

30. The method of claim 29, wherein said wire rope is a 1×7 strand wire rope.

31. The method of claim 24, wherein said spacing between each pair of said outwardly extending projections is between about 0.001 inches and about 0.1 inches.

32. The method of claim 31, wherein said spacing is about 0.06 inches.
